(19) United States
(12) Patent Application Publication
(10) Pub. No.: US 2018/0221586 A1
(43) Pub. Date: Aug. 9, 2018

(54) INJECTION DEVICE

(71) Applicant: Haselmeier AG, St. Gallen (CH)

(72) Inventor: Joachim Keitel, Esslingen (DE)

(21) Appl. No.: 15/941,667

(22) Filed: Mar. 30, 2018

Related U.S. Application Data
(63) Continuation of application No. PCT/EP2016/001599, filed on Sep. 26, 2016.

(30) Foreign Application Priority Data
Sep. 30, 2015 (DE) 202015006844.8

(51) Int. Cl. A61M 5/315; A61M 5/20

(52) U.S. Cl. CPC A61M 5/31551; A61M 5/32; A61M 5/2033; A61M 5/31585

(57) ABSTRACT

An injection device is for squeezing out a dosage of injection liquid from a container and includes: a container configured to hold the liquid; a housing defining a longitudinal central axis; first and second components; wherein the first and second component move relative to one another when the liquid is squeezed out; an injection spring configured to at least partially release stored energy to cause the dosage of the liquid to be squeezed out; a setting device to set a rate at which the liquid is squeezed out; the setting device influencing the energy required for moving the second component relative to the first; the first and second component being moveable relative to one another in the direction of the longitudinal central axis when the liquid is squeezed out; and, the setting device, when setting the rate, acting on a rotary position of the first component relative to the second.

### CROSS REFERENCE TO RELATED APPLICATIONS

This application is a continuation application of international patent application PCT/EP2016/001599, filed Sep. 26, 2016, designating the United States and claiming priority from German application 20 2015 006 844.8, filed Sep. 30, 2015, and the entire content of both applications is incorporated herein by reference.

### BACKGROUND OF THE INVENTION

An injection device which has a device for setting the injection rate is known from WO 2014/166918 A1. To this end, a latching arm which is mounted so as to be locationally fixed on the housing and which, when the dosage is squeezed out, interacts with teeth of a component that rotates about the longitudinal central axis is provided. In order for the injection rate to be set, a displacement of the component that supports the teeth is performed in the direction of the longitudinal central axis of the injection device.

### SUMMARY OF THE INVENTION

It is an object of the invention to provide an injection device which has a simple construction.

This object can, for example, be achieved by an injection device for automatically squeezing out a dosage of injection liquid from a container thereof. The injection device includes:

the container configured to hold the injection liquid; a housing defining a longitudinal central axis; a first component; a second component; wherein the first component and the second component move relative to one another when the injection liquid is squeezed out; an injection spring configured to store energy and to, when the injection liquid is squeezed out from the container, at least partially release the stored energy to cause the dosage of the injection liquid to be squeezed out from the container; a setting device to set an injection rate at which the injection liquid is squeezed out from the container; the setting device being configured to influence the energy required for moving the second component in relation to the first component; the first component and the second component being moveable relative to one another in the direction of the longitudinal central axis when the injection liquid is squeezed out; and, the setting device being configured, when setting the injection rate, to act on a rotary position of the first component in relation to the second component.

It is provided that the two components between which the setting device for setting the injection rate acts move relative to one another in the direction of the longitudinal central axis when the injection liquid is squeezed out. The setting device in the setting of the injection rate acts on the mutual relative rotary position of the two components. On account thereof, an injection device having a simple construction can be achieved, a setting of the injection rate nevertheless being possible in the case of the injection device.

The setting device preferably enables a stepless setting of the injection rate. The setting device advantageously includes an operating element which by a setting spring is pretensioned in that direction that is counter to an increase in the injection rate. On account thereof, a dynamic setting of the injection rate is possible during an injection. This is advantageous in particular when a user only rarely performs an injection and is not familiar with the injection rate that is comfortable to him/her. A dynamic setting of the injection rate can also be particularly advantageous when the volume to be injected varies from one injection to another, such that the injection rate that is perceived by the user to be the most comfortable changes from one injection to another. A dynamic setting of the injection rate can also be advantageous when the viscosity of the injection liquid to be injected depends heavily on the temperature, and dissimilar injection rates can result on account thereof at the same setting of the injection device.

A simple construction results when the setting device has a latching installation which acts between the first and the second component. The latching installation in a first rotary position of the first and of the second component has a higher force for overcoming a latching mechanism than in a second relative rotary position. A setting of the injection rate is possible in a simple manner on account thereof. The latching installation advantageously includes at least one latching element which interacts with at least one counter latching element, wherein the latching depth by which the latching elements and the counter latching elements overlap is greater in the first relative rotary position than in the second relative rotary position. The latching element is advantageously configured as a latching web which protrudes into a latching depression that forms the counter latching element. A latching web which interacts with a multiplicity of latching depressions is particularly preferably provided.

In order for a simple, intuitive operation of the setting device to be achieved, it is advantageously provided that the setting device includes a threaded connection that is formed between the second component and the housing of the injection device, and in that, for setting the injection rate, the second component is moved by the user in relation to the housing in the direction of the longitudinal central axis and by virtue of the threaded connection is rotated in relation to the housing. This is advantageous in particular when the setting of the injection rate is possible in a dynamic manner, and the operating element is pretensioned in the direction of the longitudinal central axis toward the position at which the lowest injection rate is set. The latching element advantageously runs helically about the longitudinal central axis, wherein the pitch angle of the latching element corresponds to the pitch angle of the threaded connection. On account thereof, there are no latched settings that have to be overcome when the injection rate is set. The latching element by virtue of the helical profile of the latching element remains in contact with the assigned counter latching element and moves along the assigned counter latching element when the second component by virtue of the threaded connection is moved helically in relation to the housing. No latched settings are overcome herein, and no latching noises are generated. The latching installation is active only when injection liquid is squeezed or pressed out. The setting of the injection rate, and the pressing out or squeezing out of the injection liquid, herein can be performed simultaneously. In particular, the first component moves in the direction of the longitudinal central axis when injection liquid is squeezed. If the injection rate is set or changed during the squeezing out of injection liquid, the axial movement of the first component and the helical rotating movement of the second component are superposed.

Alternatively, a setting ring which, for setting the injection rate, is to be rotated by the operator about the longitudinal central axis can also be provided for setting the injection rate. The setting ring is in particular connected to the second component. However, a connection to the first component can also be advantageous. If the setting ring is connected to the first component, the connection is advantageously configured such that the first component is rotationally fixed yet movable in the axial direction in relation to the setting ring. The injection rate is advantageously preselected by way of the setting ring prior to starting the injection, and remains at this setting during the injection. In order for an injection to be released, an operating element which, for releasing the injection, is to be displaced by the operator in the proximal direction is advantageously provided. A simple operation is achieved on account thereof. However, another device for releasing an injection can also be advantageous.

When the injection liquid is squeezed out, the first component is advantageously guided so as to be movable in the direction of the longitudinal central axis and so as to be rotationally fixed in the housing. In order for the injection rate to be set, only the second component is advantageously rotated about the longitudinal central axis. A simple construction is achieved on account thereof. The second component is advantageously a setting part of the injection device, and the first component is a metering piston which when injection liquid is squeezed out is displaced in the direction of the longitudinal central axis. The setting part advantageously has latching elements, the depth of the latter expediently varying continuously in the circumferential direction. A setting of the injection rate can be achieved in a simple manner on account thereof.

### BRIEF DESCRIPTION OF THE DRAWINGS

The invention will now be described with reference to the drawings wherein:

FIG. 1 shows a lateral view of a first embodiment of an injection device in a blocked position;

FIG. 2 shows a section along the line II-II in FIG. 1;

FIG. 3 shows the fragment III from FIG. 2 in an enlarged illustration;

FIG. 4 shows a lateral view of the injection device from FIG. 1 in a first starting position;

FIG. 5 shows a section along the line V-V in FIG. 4;

FIG. 6 shows the fragment VI from FIG. 5 in an enlarged illustration;

FIG. 7 shows a sectional illustration according to FIG. 5 in a terminal position of the injection device;

FIG. 8 shows the fragment VIII from FIG. 7 in an enlarged illustration;

FIG. 9 shows the injection device in a second starting position, in a lateral view;

FIG. 10 shows a section along the line X-X in FIG. 9;

FIG. 11 shows the fragment XI from FIG. 10 in an enlarged illustration;

FIG. 12 shows a perspective illustration of the metering piston of the injection device;

FIG. 13 shows a lateral view of the metering piston;

FIG. 14 shows a lateral view in the direction of the arrow XIV in FIG. 13;

FIG. 15 shows a section along the line XV-XV in FIG. 14;

FIGS. 16 and 17 show perspective illustrations of a setting part of the injection device;

FIG. 18 shows a lateral view of the setting part;

FIG. 19 shows a section along the line IX-IX in FIG. 18;

FIG. 20 shows a lateral view in the direction of the arrow XX in FIG. 19;

FIG. 21 shows a lateral view of an upper housing part of the injection device;

FIG. 22 shows a section along the line XXII-XXII in FIG. 21;

FIGS. 23 and 24 show perspective illustrations of an operating element of the injection device;

FIG. 25 shows a lateral view of the operating element;

FIG. 26 shows a section along the line XXVI-XXVI in FIG. 25;

FIG. 27 shows a lateral view of a lower housing part of the injection device;

FIG. 28 shows a section along the line XXVIII-XXVIII in FIG. 27;

FIG. 29 shows a lateral view of a further embodiment of an injection device in a blocked position, at a first set injection rate;

FIG. 30 shows a section along the line XXX-XXX in FIG. 29;

FIG. 31 shows the fragment XXXI-XXXI from FIG. 30 in an enlarged illustration;

FIG. 32 shows a lateral view of the injection device, at a second set injection rate;

FIG. 33 shows a section along the line XXXIII-XXXIII in FIG. 32;

FIG. 34 shows an enlarged illustration of the fragment XXXIV from FIG. 33;

FIG. 35 shows a lateral view of the injection device at a third set injection rate;

FIG. 36 shows a section along the line XXXVI-XXXVI in FIG. 35;

FIG. 37 shows the fragment XXXVII from FIG. 36 in an enlarged illustration;

FIG. 38 shows a lateral view in the direction of the arrow XXXVIII in FIG. 35;

FIG. 39 shows a section along the line XXXIX-XXXIX in FIG. 38;

FIG. 40 shows an enlarged illustration of the fragment XL in FIG. 39;

FIG. 41 shows a lateral view of the injection device in a starting position;

FIG. 42 shows a section along the line XLII-XLII in FIG. 41;

FIG. 43 shows the fragment XLIII from FIG. 42 in an enlarged illustration;

FIGS. 44 and 45 show perspective illustrations of the metering piston of the injection device;

FIG. 46 shows a lateral view of the metering piston;

FIG. 47 shows a lateral view of the metering piston in the direction of the arrow XLVII in FIG. 46;

FIG. 48 shows a section along the line XLVIII-XLVIII in FIG. 47;

FIG. 49 shows the fragment IL in FIG. 48 in an enlarged illustration;

FIG. 50 shows a lateral view of the setting part;

FIG. 51 shows a section along the line LI-LI in FIG. 50;

FIG. 52 shows a plan view of the setting part in the direction of the arrow LII in FIG. 51;

FIG. 53 shows a section along the line LIII-LIII in FIG. 51;

FIG. 54 shows a lateral view of the upper housing part of the injection device;

FIG. 55 shows a section along the line LV-LV in FIG. 54;

FIG. 56 shows a section along the line LVI-LVI in FIG. 55;

FIG. 57 shows a section along the line LVII-LVII in FIG. 55;

FIGS. 58 and 59 show perspective illustrations of the operating element;

FIG. 60 shows a lateral view of the operating element;

FIG. 61 shows a section along the line LXI-LXI in FIG. 60;

FIG. 62 shows a lateral view of the lower housing part of the injection device; and,

FIG. 63 shows a section along the line LXIII-LXIII in FIG. 62.

### DESCRIPTION OF THE PREFERRED EMBODIMENTS OF THE INVENTION

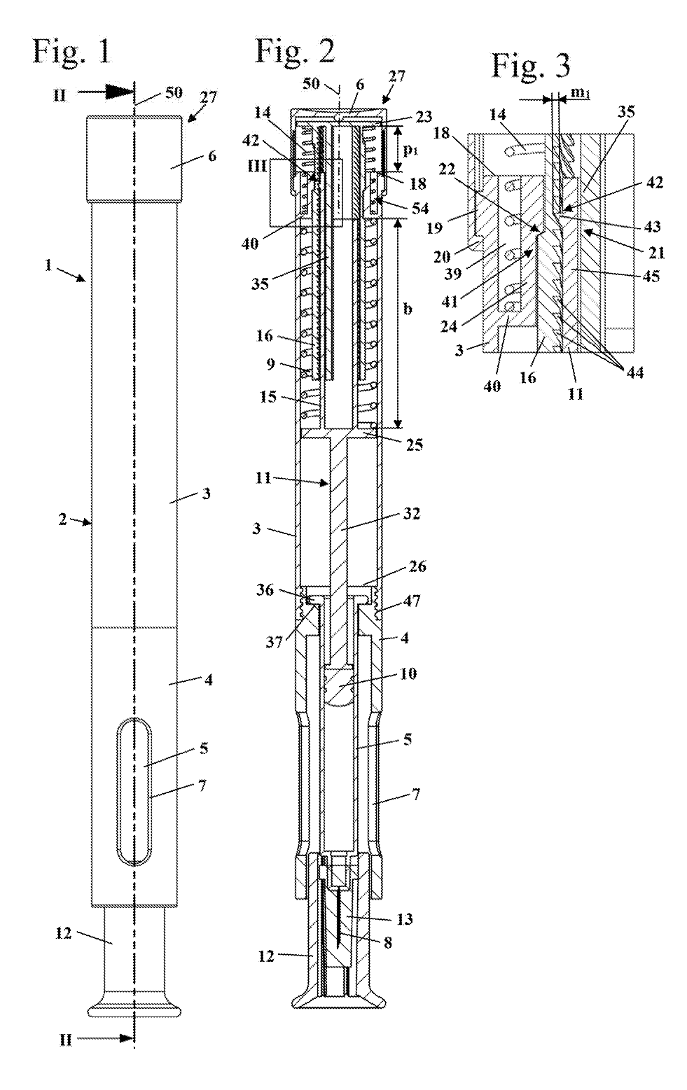

FIG. 1 shows an injection device 1 having a housing 2. The injection device 1 is provided for squeezing out a one-shot dosage of injection liquid from a container 5 that is disposed in the housing 2. The injection device 1 is subsequently disposed of. Injection devices of this type are also referred to as autoinjectors. The housing 2 is constructed from an upper distal housing part 3, and from a lower proximal housing part 4. Two viewing windows 7, one of which being visible in FIG. 1, are disposed so as to be mutually opposite in the lower housing part 4. The container 5 that is disposed in the housing 2 is visible through the viewing window 7. A safety cap 12 which covers an injection needle 8 that is visible in FIG. 2 is held at the proximal end of the housing 2. An operating element 6 is disposed at the distal end of the housing 2. The injection device in FIGS. 1 to 3 is shown in a blocked position 27 in which the squeezing out of injection liquid from the container 5 is blocked.

The container 5 is configured as a syringe, as is shown in FIG. 2. The injection needle 8 is held at the proximal end of the container 5. The injection needle 8 is disposed in a needle protector 13 which covers the injection needle 8 such that the injection needle 8 remains sterile. The safety cap 12 is fixedly connected to the needle protector 13 such that the needle protector 13 is also pulled off when the safety cap 12 is pulled off. A protective sleeve which is pushed back only when the injection needle 8 pierces the skin can additionally be provided. As is also shown in FIG. 2, the container 5 at the distal end thereof has an outwardly protruding periphery 36. The lower housing part 4 has a shoulder 37 on which the periphery 36 of the container bears. On account thereof, the container 5 cannot move in the proximal direction.

The injection device 1 has a metering piston 11, as is shown in FIG. 2. The metering piston 11, in a proximal manner, has a piston rod 32 which acts on a plug 10 of the container 5 and moves the latter in the proximal direction in order for injection liquid to be squeezed out. The metering piston 11, in a distal manner, has a sleeve portion 15. A periphery 25 which is aligned so as to be perpendicular to a longitudinal central axis 50 of the injection device 1 extends between the sleeve portion 15 and the piston rod 32 in the embodiment shown, an end of an injection spring 9 bearing on the periphery 25. The injection spring 9 is configured as a compression spring, specifically as a compression coil spring, and by way of the second distal end thereof is supported on a wall portion 40 of the upper housing part 3. The injection spring 9 is disposed in an annular space which is formed between the upper housing part 3 and a setting part 16 that is disposed in the housing 2. The injection spring 9 is already tensioned in the production of the injection device 1. The injection spring 9 serves for moving the metering piston 11 in the proximal direction, and on account thereof for squeezing out injection liquid. The metering piston 11 in the embodiment shown, by way of guide means (not shown) is guided in a rotationally fixed manner in the housing 2. As is also shown in FIG. 2, the housing parts 3 and 4 are interconnected at a threaded connection 47. The distal end side of the threaded connection 47 forms a shoulder which forms a detent 26 for the proximal terminal position of the metering piston 11. The periphery 25 of the metering piston 11 in the proximal terminal position of the latter bears on the detent 26.

The setting part 16 is mounted in the housing 2 so as to be rotatable about the longitudinal central axis 50. Moreover, the setting part 16 is axially movable by a predefined distance in the direction of the longitudinal central axis 50. The setting part 16 is part of a setting device 41 for setting the injection rate. The setting part 16 is configured so as to be sleeve-shaped and is disposed on the external circumference of the sleeve portion 15 of the metering piston 11. The setting part 16 on the distal side thereof has a bearing periphery 23 on which the operating element 6 in the embodiment bears. A setting spring 14 which in the embodiment is configured as a compression coil spring and which pushes the operating element 6 and the setting part 16 in the distal direction acts between the upper housing part 3 and the bearing periphery 23 of the operating element 6. In the blocked position 27 shown in FIGS. 1 to 3, the bearing periphery 23 has a spacing p₁, measured in the direction of the longitudinal central axis 50 from the bearing periphery 18 of the upper housing part 3, which corresponds to the maximal spacing between the bearing peripheries 18 and 23. The bearing periphery 18 in the embodiment is configured on the distal end side of the housing 2 and conjointly with the bearing periphery 23 forms a detent for the proximal position of the operating element 6.

The setting part 16 by way of a threaded connection 22 is mounted so as to be rotatable and axially displaceable in the upper housing part 3. A latching device 42 is provided between the setting part 16 and the sleeve portion 15 of the metering piston 11. As is shown in the enlarged illustration in FIG. 3, the metering piston 11 has a latching portion 45 on which a radially outwardly protruding latching web 43 is configured. The latching web 43 protrudes into a latching depression 44 which is provided on the internal side of the setting part 16. As is shown in FIG. 3, a multiplicity of latching depressions 44 are provided. In an axial movement of the metering piston 11 in the proximal direction, the latching web 43 conjointly with the latching depressions 44 forms the latching installation 42. The latching installation 42 applies a force counter to the movement of the metering piston 11 in the proximal direction. A support portion 35 is provided on the radially inward side of the latching portion 45 in the blocked position 27 shown in FIGS. 1 to 3. The support portion 35 is at a minor radial spacing from the latching portion 45 and prevents the latching portion 45 from being able to move in a radially inward manner, thus in the direction toward the longitudinal central axis 50. On account thereof, the latching web 43 cannot move out of the assigned latching depression 44. Any movement of the metering piston 11 in the proximal direction is blocked on account thereof. The support portion 35 conjointly with the latching arm 45 forms a blocking installation 21 which prevents the injection spring 9 from being able to move the metering piston 11 in the proximal direction.

As is also shown in FIG. 3, the threaded connection 22 is formed between the external circumference of the setting part 16 and the internal side of a threaded portion 24 of the upper housing part 3. An annular space 39, the base thereof forming the wall portion 40, is formed between the threaded portion 24 and the external wall of the upper housing part 3. The setting spring 14 is also supported on the wall portion 40. The setting spring 14, by way of the other end thereof, is supported on the bearing periphery 23 of the setting part 16, as is shown in FIG. 2.

In the blocked position 27, the operating element 6 is in the distal terminal position thereof. Guide cams 19, one of which being shown in FIG. 3, are configured on the upper housing part 3. The guide cams 19 interact with a holding periphery 20 of the operating element 6, and hold the operating element 6 on the upper housing part 3. In the blocked position 27, the latching web 43 by way of a latching depth m₁ engages in a latching depression 44. The latching depth m₁ describes the overlap between the latching web 43 and the latching depression 44 in the radial direction in relation to the longitudinal central axis 50. The latching depth ml is comparatively great. In the blocked position 27, the setting part 16 and the metering piston 11 have a mutually relative rotary position 54. The support portion 35 in this relative rotary position 54 is disposed on the latching portion 45 and, on account thereof, prevents any movement of the latching web 43 out of the latching depression 44 and, on account thereof, any movement of the metering piston 11 in the proximal direction.

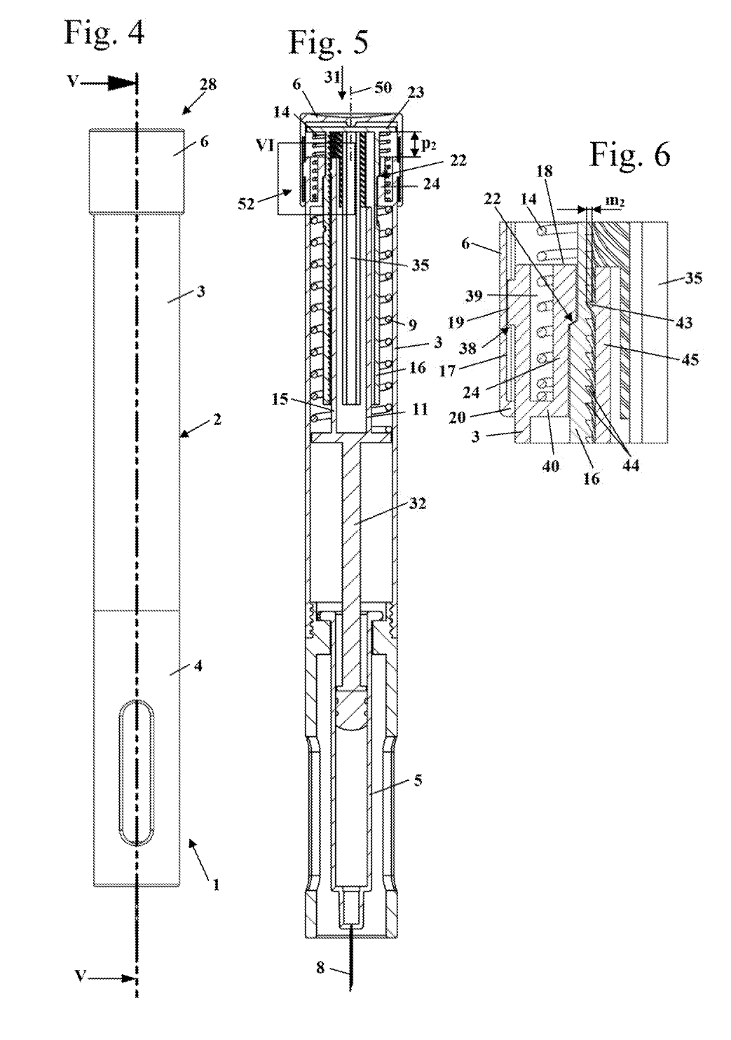

In order for an injection to be performed, the operating element 6 is pushed by the operator in the proximal direction 31, as is schematically illustrated in FIG. 5. FIGS. 4 to 6 show the injection device 1 in a first starting position 28 at which a first, comparatively minor, injection rate is set. In relation to the blocked position 27 shown in FIGS. 1 to 3, the operating element 6 has been moved in the proximal direction 31. On account thereof, the spacing between the bearing periphery 23 and the bearing periphery 18 of the upper housing part 3 has been reduced from the spacing p₁ to a spacing p₂. The operating element 6 has displaced the setting part 16 in the proximal direction 31. The setting part 16, by virtue of the threaded connection 22, herein has been rotated about the longitudinal central axis 50 in relation to the housing 2. The metering piston 11 is guided in a rotationally fixed manner in the housing 2, for example by way of respective guide webs or the like, which are not illustrated in the figures. By virtue of the movement of the operating element 6 and of the setting part 16 in the proximal direction 31, the relative rotary position of the setting part 16 in relation to the metering piston 11 is changed by virtue of the threaded connection 22. In the first starting position 28, the setting part 16 and the metering piston 11 are in a first relative rotary position 52 which is assigned to the first injection rate. As is shown in FIG. 6, the latching depressions 44 are helically configured. The pitch of the latching depressions 44 herein corresponds to the pitch of the threaded connection 22. On account thereof, the latching web 43 in the helicoid movement of the setting part 16 in relation to the housing 2 and in relation to the metering piston 11 remains in the same latching depression 44 such that no latching noises are generated.

The latching depressions 44 in different regions of the circumference of the setting part 16 have dissimilar depths. In the starting position 28 shown in FIGS. 4 to 6, the latching web 43 is disposed in a region of the latching depressions 44 in which the latching depressions 44 have a reduced depth in comparison to the arrangement in the blocked position 27. The latching depth m₂, measured in the radial direction in relation to the rotation axis 50, by way of which the latching web 43, when viewed in the direction of the longitudinal central axis 50, overlaps the latching depressions 44 is smaller than the latching depth m₁, shown in FIG. 3, in the blocked position 27. However, the latching depths m₁ and m₂ can also be of identical size, since no squeezing out of injection liquid is possible in the blocked position 27. As is shown in FIGS. 5 and 6, the support portion 35 by virtue of the rotating movement of the setting part 16 in relation to the metering piston 11 has moved away from the latching portion 45. On account thereof, the latching portion 45 by virtue of the force exerted by the injection spring 9 can be deflected in a radially inward manner from the inclined lateral walls of the latching depressions 44. On account thereof, a movement of the metering piston 11 in the proximal direction 31 is possible. In the movement of the metering piston 11 in relation to the setting part 16, the latching web 43 latches across the elevations formed between the latching depressions 44, such that latching steps are audible.

As is also shown in FIG. 6, the guide cam 19 protrudes into a groove 17 of the operating element 6 and conjointly with the latter forms a guide 38 which guides the operating element 6 in the direction of the longitudinal central axis 50 on the upper housing part 3, preventing any rotation of the operating element 6 in relation to the upper housing part 3.

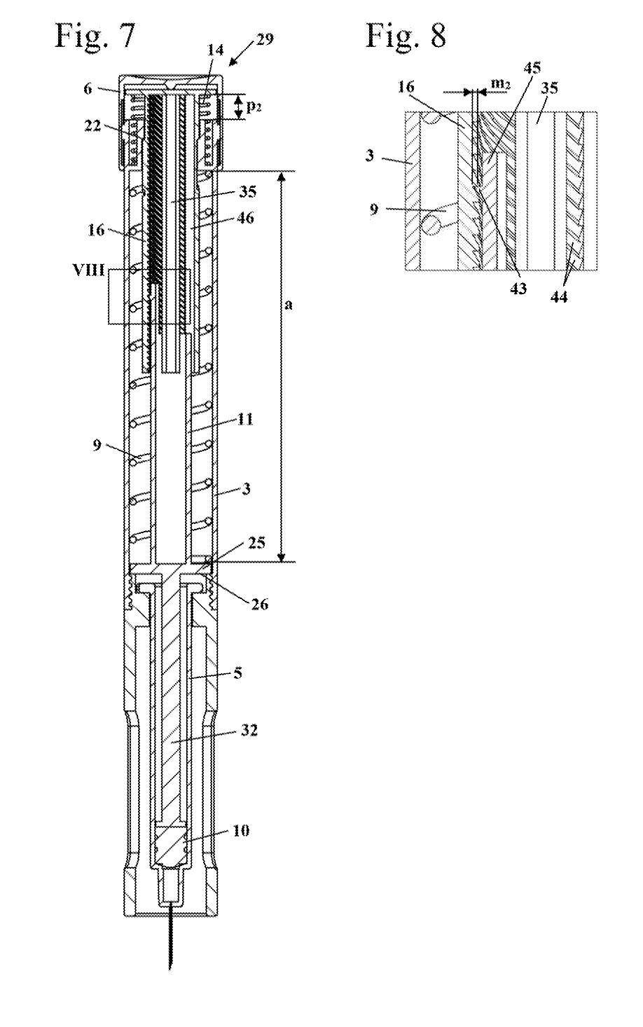

FIGS. 7 and 8 show the injection device 1 after the injection, in a terminal position 29. To this end, the operator has adjusted the operating element 6 to the position shown in FIG. 6. The injection starts once the support portion 35 has released a movement of the latching portion 45, this by virtue of the force stored in the injection spring 9 being automatic. In the embodiment shown, the operator has held the operating element 6 in the same position as in FIGS. 4 to 6 until the end of the injection. On account thereof, the same latching depth m₂ has resulted for the latching web 43 during the entire injection procedure, such that the force which the latching installation 42 has directed counter to the force exerted by the injection spring 9 on the metering piston 11 has been constant. The injection rate during the injection procedure can be set dynamically by displacing the operating element 6 further in the proximal direction 31, or by yielding at the operating element 6 such that the setting spring 14 moves the operating element 6 in the distal direction. The injection ends when the periphery 25 comes to bear on the detent 26.

The first relative rotary position 52 and the second relative rotary position 53 are relative rotary positions at which an injection can be performed. In the third relative rotary position 54, shown in FIGS. 1 to 3, the injection device 1 is in the blocked position 27 thereof, and no injection can be performed.

FIG. 2 shows the injection spring 109 at the tensioned length b thereof. After the injection, the injection spring 9 has extended in length to the non-tensioned length a thereof shown in FIG. 7. The difference between the non-tensioned length a and the tensioned length b corresponds to the distance which is traveled by the metering piston 11 when the injection liquid is squeezed out.

The configuration of the latching depressions 44 is also shown in FIGS. 7 and 8. As is shown in FIG. 7, the metering member 16 has a wall portion 46 without latching depressions 44, the wall portion 46 extending across a circumferential portion and the entire length of the metering member 16.

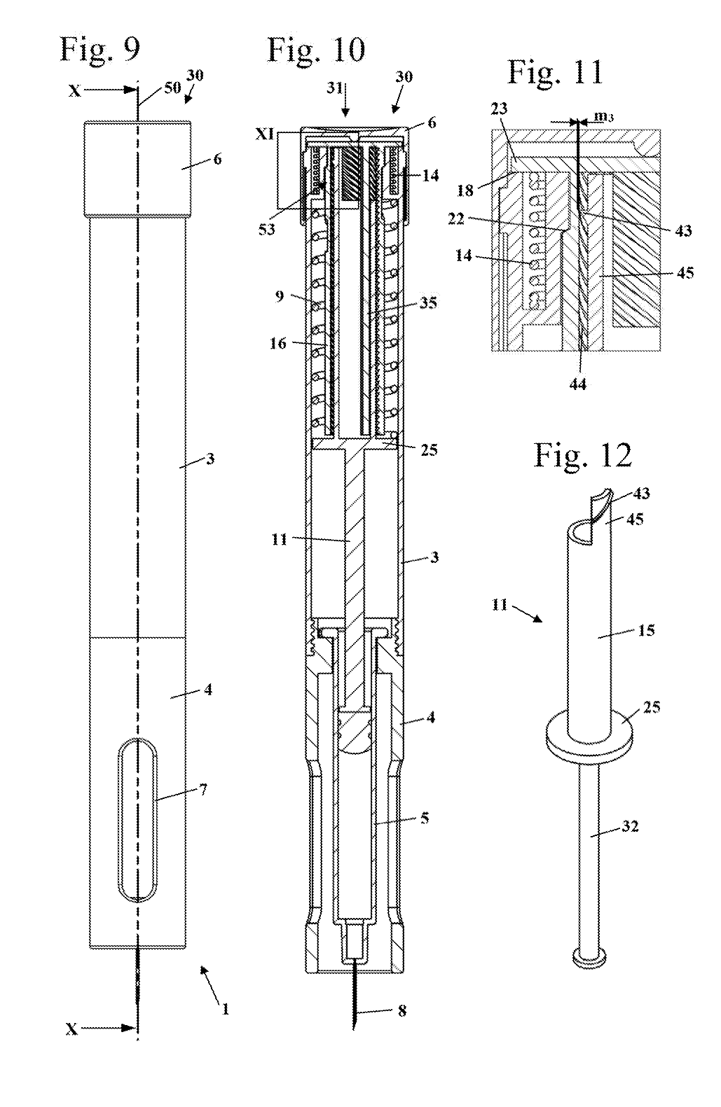

FIGS. 9 to 11 show the injection device 1 in a starting position 30 at which a high injection rate is set. In the starting position 30 the operating element 6 is in the proximal terminal position thereof. In the proximal terminal position, the latching web 43 engages in the latching depressions 44 by way of only a minimal latching depth m₃. The latching depth m₃ can also be zero. The latching web 43 can also be disposed in the wall portion 46, shown in FIG. 7, in which no latching depressions 44 are disposed. The starting position 30 has been attained by moving the operating element 6 in the proximal direction. By virtue of the movement of the operating element 6 and of the setting part 16 in the proximal direction, the setting part 16 by virtue of the threaded connection 22 has rotated about the longitudinal central axis 50. The setting part 16 and the metering piston 11 are in a second relative rotary position 53 at which the latching depth m₃ is less than at the relative rotary position 52. On account thereof, the injection rate at the second relative rotary position 53 is greater than at the first relative rotary position 52. By virtue of the setting spring 14, the operator can change the injection rate dynamically during the injection by enlarging or reducing the pressure on the operating element 6. The distal bearing periphery 23 of the setting part 26 in the terminal position shown in FIGS. 9 to 11 bears on the distal bearing periphery 18 of the upper housing part 3.

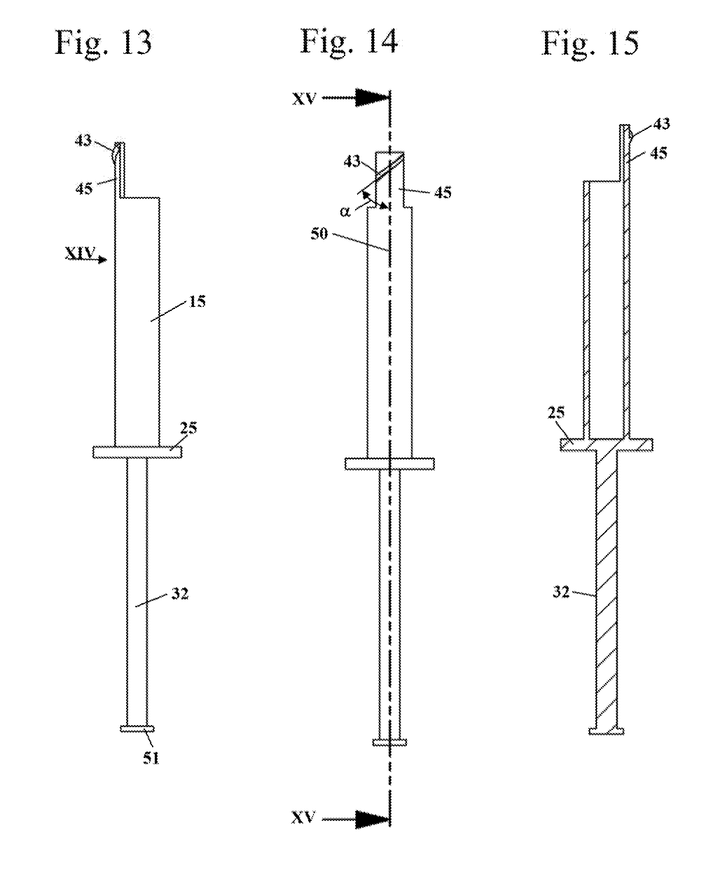

FIGS. 12 and 13 show the metering piston 11 in a perspective illustration. The latching portion 45 protrudes from the sleeve-shaped portion 15 in the distal direction, and extends only across a part-region of the circumference. The desired stiffness of the latching portion 45 can be set by way of the extent of the latching portion 45 in the circumferential direction. A bearing disk 51 which is configured for bearing on the plug 10 (FIG. 2) is disposed on the proximal end on the piston rod 32.

As is shown in FIG. 14, the latching web 43 in a lateral view is inclined in relation to the longitudinal central axis 50 by an angle α. The angle a corresponds to the pitch angle of the threaded connection 22. On account thereof, the latching web 43 in the rotation of the setting part 16 about the longitudinal central axis 50 is guided helically in a single latching depression 44. The latching web 43 is configured so as to be triangular in the cross section, as is shown in FIG. 15.

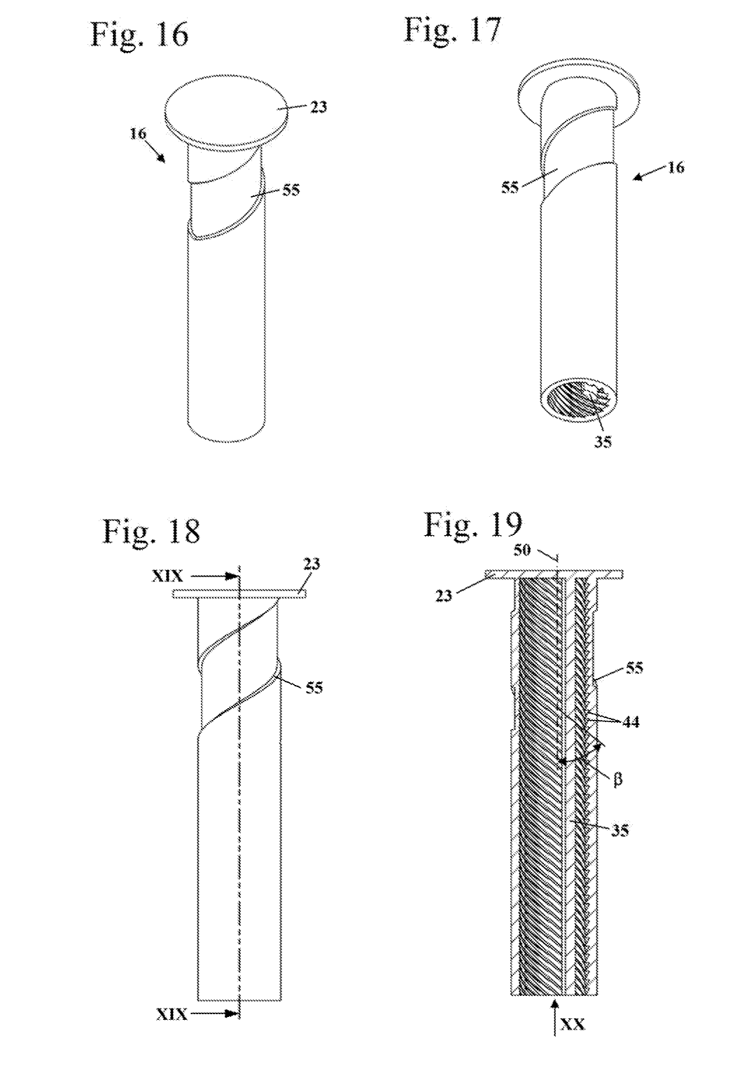

FIGS. 16 to 20 show the setting part 16 in detail. The setting part 16 is configured so as to be sleeve-shaped and at the distal end thereof has the bearing periphery 23 which in the embodiment closes the interior space of the setting part 16 in the distal direction. An external thread 55 is configured on the external side of the setting part 16. The external thread 55 in the embodiment extends by less than one revolution.

As is shown in FIG. 19, the latching depressions 44 are inclined in relation to the longitudinal central axis 50 by an angle β which corresponds to the angle a (FIG. 14). The latching web 43 in the manner of a thread engages in a latching depression 44. As is also shown in FIG. 19, the latching depressions 44 extend across the entire axial length of the sleeve-shaped body of the metering part 16.

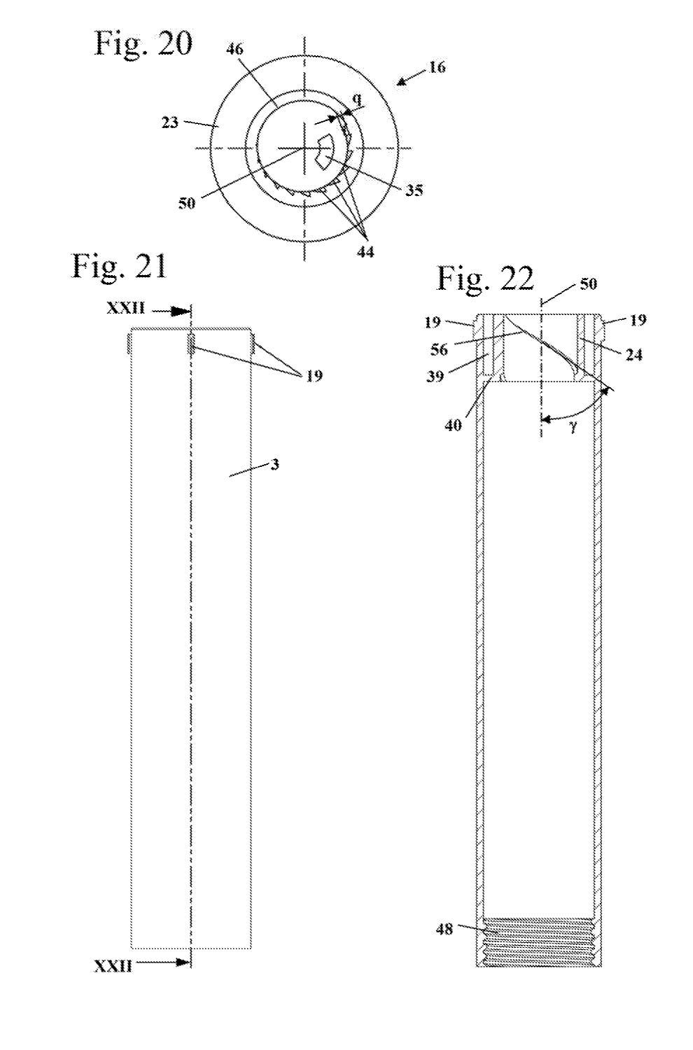

FIG. 20 shows the configuration of the latching depressions 44 in detail. The latching depressions 44 have a depth q which decreases across the circumference. On account thereof, the latching depth can be set by rotating the metering part 16 about the longitudinal central axis 50. As is also shown in FIG. 20, the support portion 35 extends so as to be parallel with the longitudinal central axis 50 and so as to be adjacent to the latching depressions 44 having the greatest depth q. The wall portion 46 which does not support any latching depressions 44 is also shown in FIG. 20. However, it can also be provided that the latching depressions 44 extend across the entire circumference of the setting part 16.

FIGS. 21 and 22 show the upper housing part 3. Four guide cams 19 are provided at the distal end of the upper housing part 3 in the embodiment. FIG. 22 also shows the configuration of the threaded portion 24 which runs radially within the external wall of the upper housing part 3 and delimits the annular space 39. An internal thread 56 which conjointly with the external thread 55 of the setting part 16 forms the threaded connection 22 is configured in the threaded portion 24. The threaded connection 22, when viewed in the lateral view, has a pitch angle y which is measured in relation to the longitudinal central axis 50 and which corresponds to the angles α and β of the latching installation 42. The upper housing part 3 has an internal thread 48 at the proximal end. The internal thread 48 serves for the connection to the lower housing part 4.

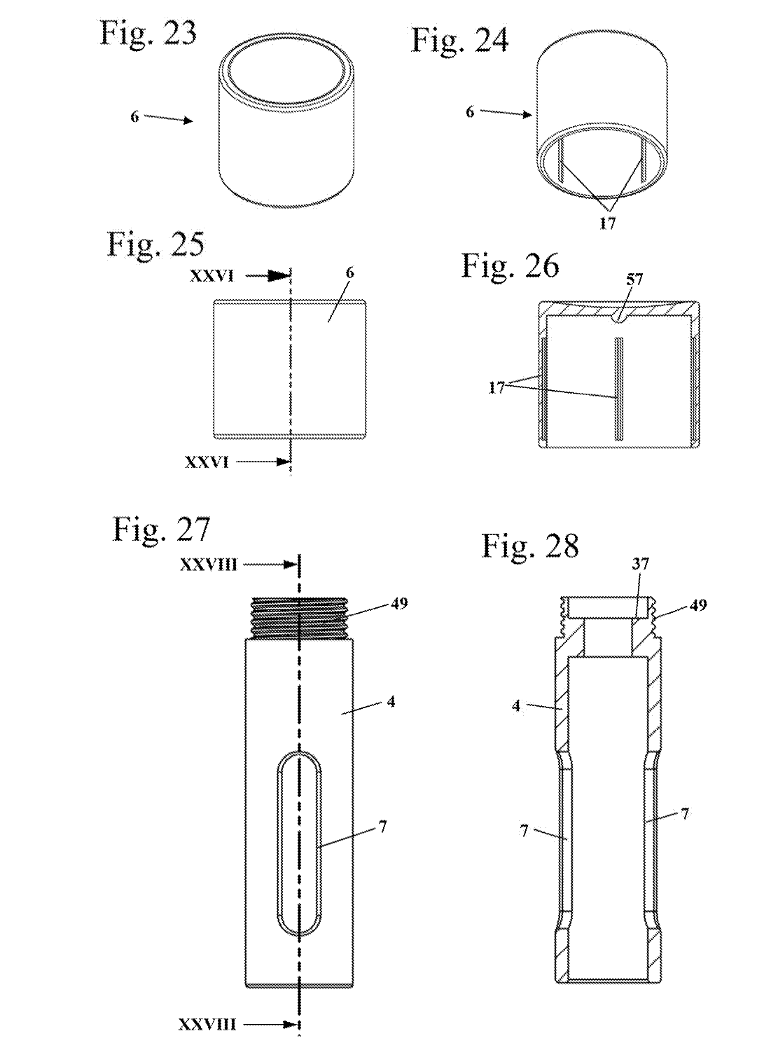

FIGS. 23 to 26 show the operating element 6 in detail. The operating element 6 is configured so as to be approximately pot-shaped. The grooves 17 on the internal side of the circumferential wall of the operating element 6 are visible in FIGS. 24 and 26. The operating element 6 on the end side thereof has an inwardly protruding protrusion 57 which is provided for bearing on the bearing periphery 23 of the setting part 16.

FIGS. 27 and 28 show the lower housing part 4 having the two viewing windows 7. The lower housing part 4 at the distal end has an external thread 49 which conjointly with the internal thread 48 of the upper housing part 3 forms the threaded connection 27. However, the housing parts 3 and 4 can also be interconnected in another manner. The shoulder 37 which serves as a bearing for the periphery 36 of the container 5 is configured at the distal end on the internal side of the lower housing part 4.

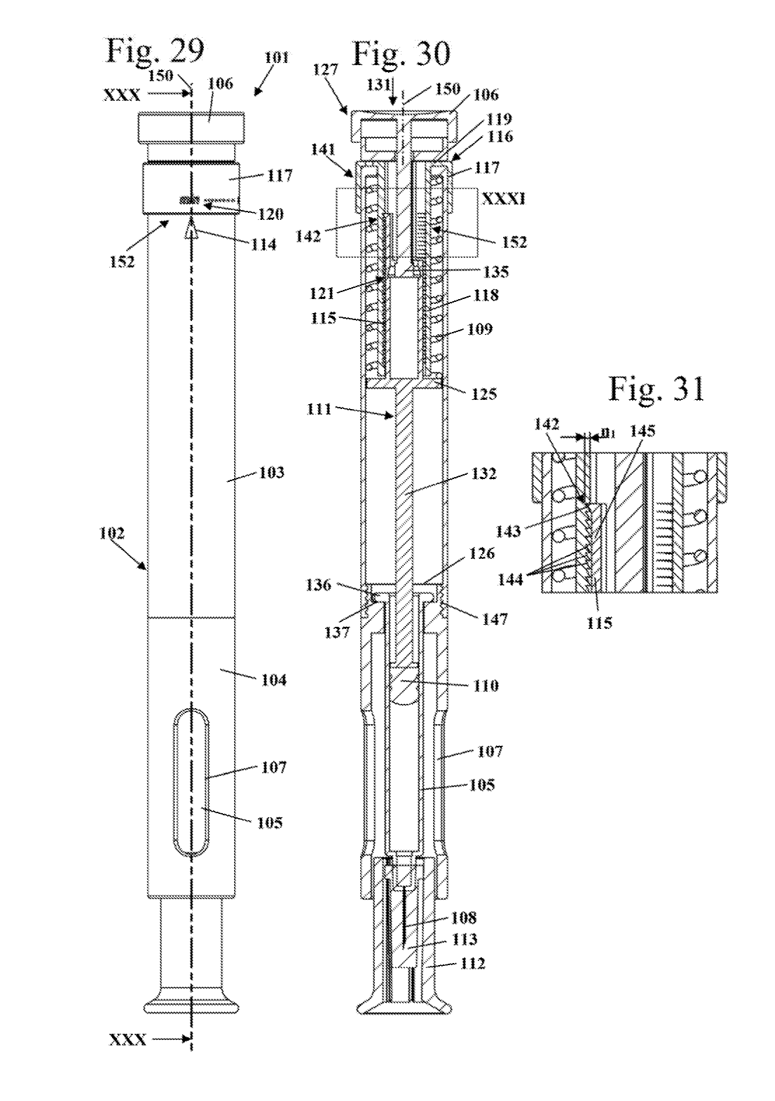

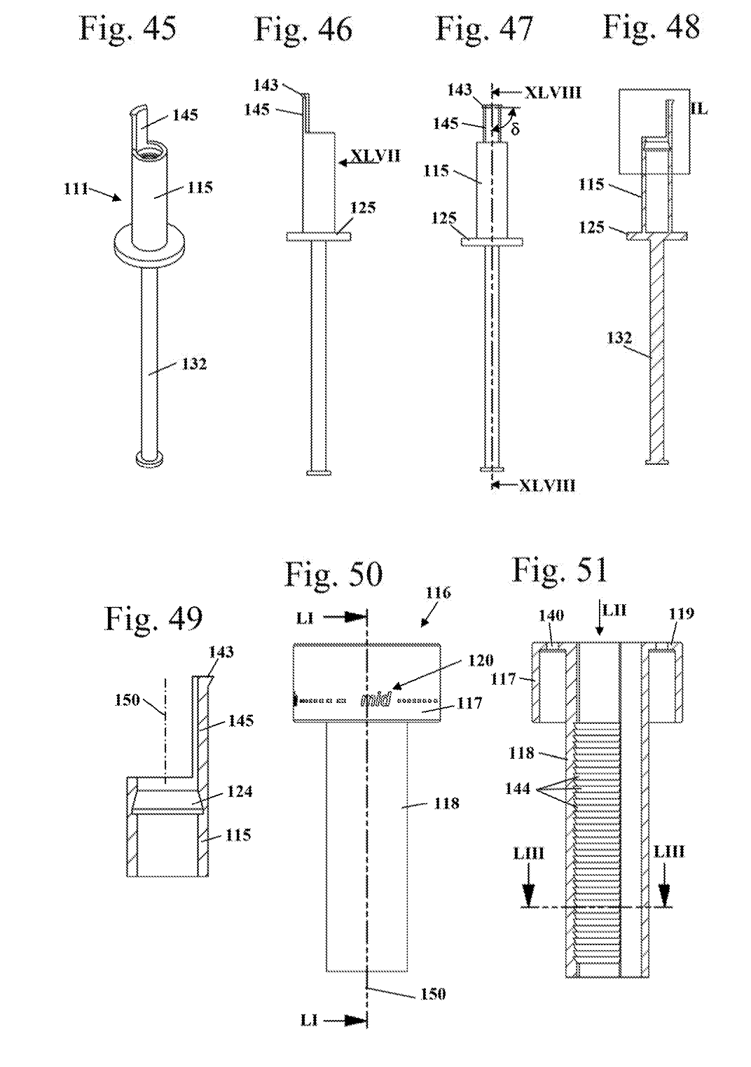

FIGS. 29 to 63 show an embodiment of an injection device 101. The injection device 101 is also an autoinjector which serves for squeezing out a one-shot dosage from a container 105, in particular from a syringe. The injection device 101 has a housing 102 which is constructed from an upper housing part 103 and from a lower housing part 104. Viewing windows 107 through which a container 105 is visible are provided in the lower housing part 104. An operating element 106 which is displaceable in the direction of a longitudinal central axis 150 of the injection device 101 is provided at the distal end of the housing 102. Moreover, a setting ring 117 is rotatably mounted on the upper housing part 103. Lettering 120 which indicates the set injection rate is provided on the setting ring 117 in the embodiment. The upper housing part 103 has a marking 114 which points toward the set dosage.

As is shown in FIG. 30, the container 105 is configured as a syringe which by way of the distal periphery 136 thereof bears on a shoulder 137 of the housing 102. A metering piston 111 which has a piston rod 132 which protrudes in the proximal direction 131 is disposed in the housing 102. The piston rod 132 by way of the proximal end thereof bears on a plug 110 of the container 105. An injection needle 108 which is covered by a needle protector 113 and by a safety cap 112 is disposed on the container 105. An injection spring 109 which is pretensioned in the production of the injection device 101 is disposed in the housing 102. The injection spring 109 is supported on a periphery 125 of the metering piston 111 and on a proximal wall of the upper housing part 103. The metering piston 11 has a sleeve portion 115 which protrudes in a distal direction, a sleeve portion 118 of a setting part 116 being disposed on the external circumference of the sleeve portion 115. The sleeve portion 115 on the distal side thereof has a latching portion 145 which is shown enlarged in FIG. 31. The latching portion 145 has a latching web 143 which protrudes into a latching depression 144 on the internal circumference of the setting part 116. The latching web 143 conjointly with the latching depressions 144 forms a latching installation 142.

The setting ring 117 is configured so as to be integral to the sleeve portion 118 and conjointly with the latter forms the setting part 116. As is shown in FIG. 30, the setting ring 117 engages across the upper housing part 103 on the distal side thereof. The metering piston 111 and the setting part 116 form a setting device 141 for setting the injection rate. The relative rotary position of the metering piston 111 and of the setting part 116 is changed by rotating the setting part 116 at the setting ring 117, and the injection rate is set on account thereof. The latching device 142 is part of the setting device 141. The operating element 106 is configured so as to be integral to a support portion 135, the configuration and function of which are yet to be explained in more detail hereunder. In the first relative rotary position 152 of the metering piston 111 and of the setting part 116, shown in FIGS. 29 to 31, a minimal injection rate is set. The latching installation 142 in the first rotary position 152 has a latching depth n₁ which is comparatively great. In an injection, the metering piston 111 in relation to the setting part 116 moves in the proximal direction. The latching web 143 herein has to overcome the individual latching depressions 144. By virtue of the comparatively great latching depth n₁, the force which is generated herein and acts counter to the force of the injection spring 109 is comparatively large such that a minor injection rate results.

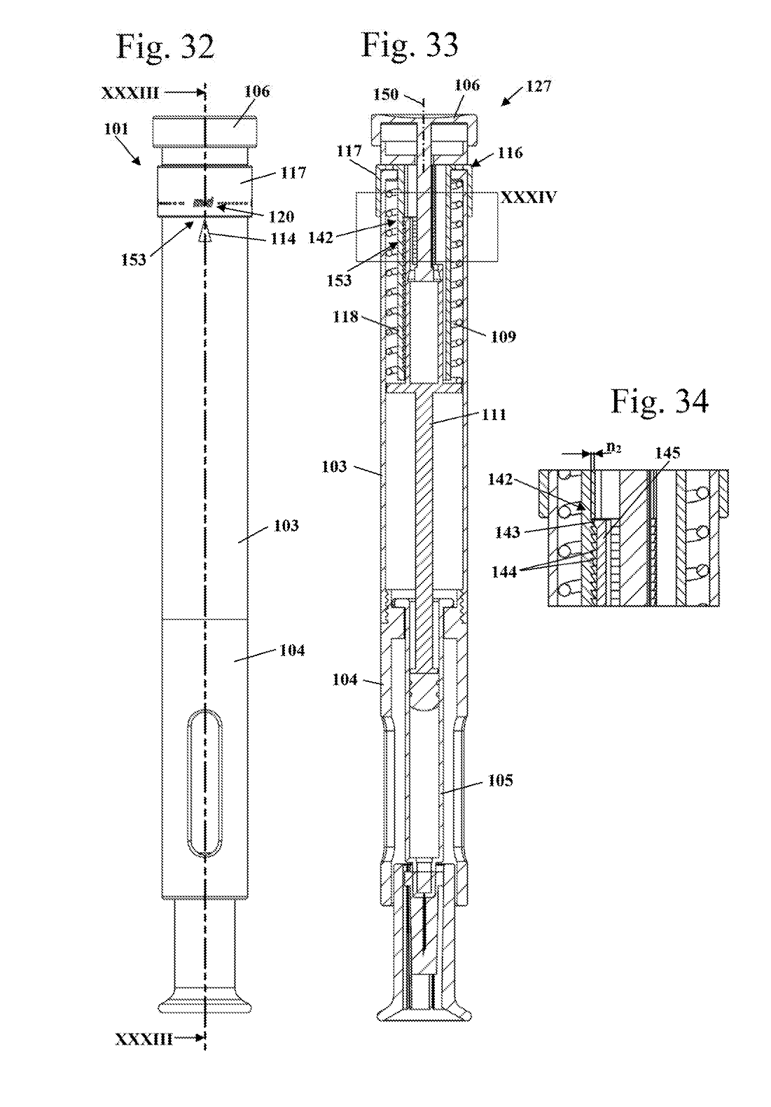

FIGS. 32 to 34 show the injection device 101 in the second relative rotary position 153 of the metering piston 111 and of the setting part 116. A medium injection rate is set in the rotary position 153, as is also shown by the lettering 120. As is shown in FIG. 34, the latching web 143 engages in the latching depression 144 by way of a latching depth n₂. The latching depth n₂ is smaller than the latching depth n₁, such that the force which is provided by the latching device 142 counter to a movement of the metering piston 111 in the proximal direction is reduced in relation to the first relative rotary position 152. As is shown in FIG. 34, the latching depressions 144 run in each case in planes that are perpendicular to the longitudinal central axis 150. In the change of the set injection rate, the setting ring 117 and thus also the sleeve portion 118 of the setting part 116 are rotated only about the longitudinal central axis 150. No movement takes place in the direction of the longitudinal central axis 150. On account thereof, the latching web 143 in the change of the injection rate remains in the same latching depression 144. The latching web 143 slides from the latching depression 144 to the latching depression 144 only in the case of the movement of the metering piston 111 in the proximal direction 131, herein generating clicking noises and a force that acts counter to the force of the injection spring 109.

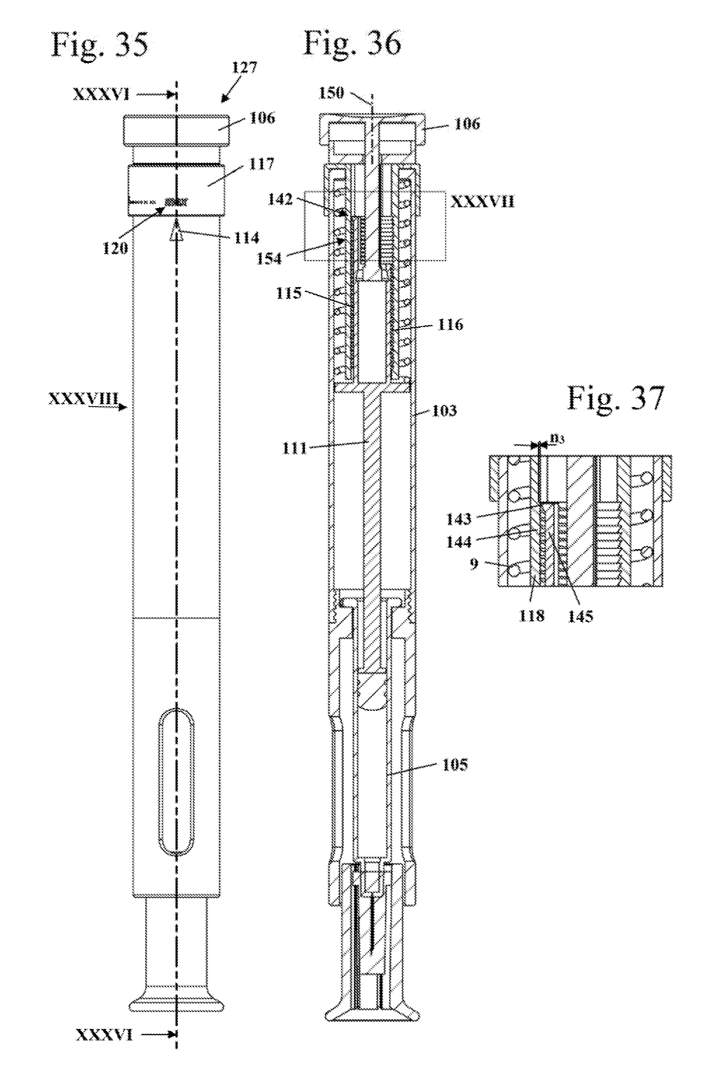

FIGS. 35 to 37 show the injection device 101 in a third relative rotary position 154 of the metering piston 111 and of the setting part 116, at which the maximum injection rate is set. The operating button 106 continues to remain in the distal terminal position thereof. No injection can take place on account thereof, and the injection device 101 is in the locked position 127 thereof. As is shown in FIG. 37, the latching web 143 protrudes only slightly into the latching depression 144. The latching depth n₃ is very minor. It can also be provided that the latching web 143 does not protrude into a latching depression 144 but in the direction of the longitudinal central axis 150 is freely movable in relation to the sleeve portion 118 of the setting part 116.

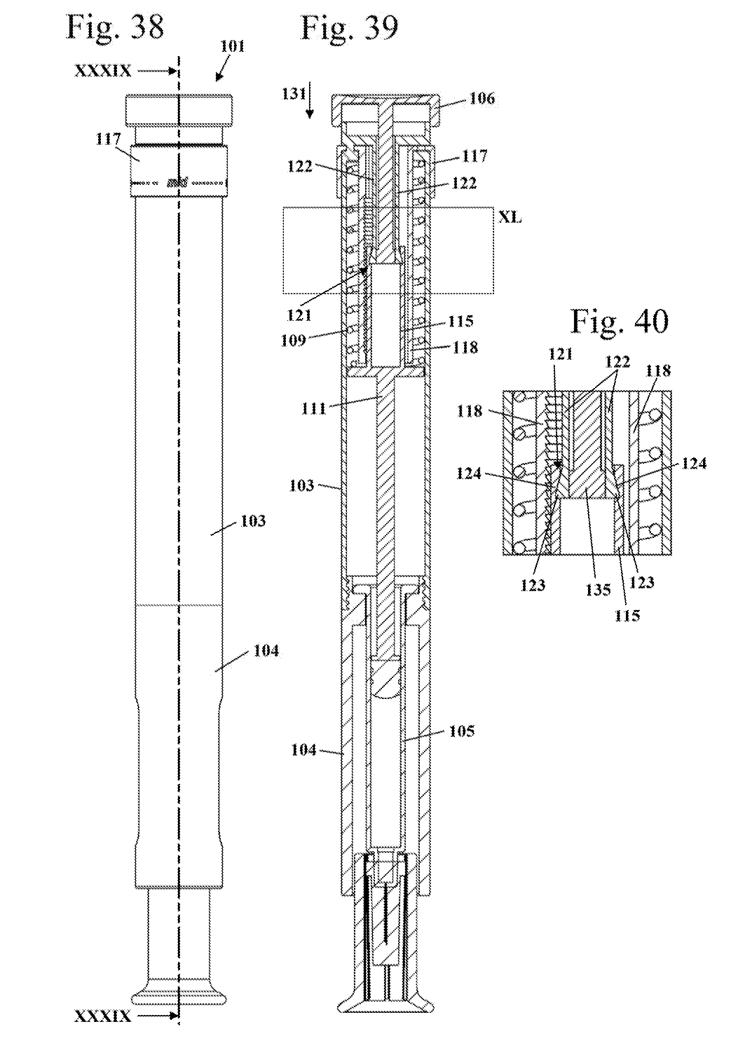

FIGS. 38 to 40 show a blocking installation 121 of the injection device 101 in detail. The blocking installation 121 prevents that the metering piston 111 in the distal terminal position of the operating element 106 is able to move in the proximal direction and herein is able to squeeze out injection liquid. As is shown in FIG. 39, two locking arms 122 which protrude into the sleeve portion 115 of the metering piston 111 are provided on the upper housing part 103. As is shown in FIG. 40, the locking arms 122 at the proximal ends thereof support outwardly protruding locking protrusions 123 which engage in at least one locking depression 124 of the sleeve portion 115 of the metering piston 111. The blocking portion 135 is disposed radially within the locking arms 22 and prevents that the locking arms 122 are able to be deflected in a radially inward manner. The locking arms 122 fix the sleeve portion 115 by way of the locking protrusions 123 and of the locking depressions 124.

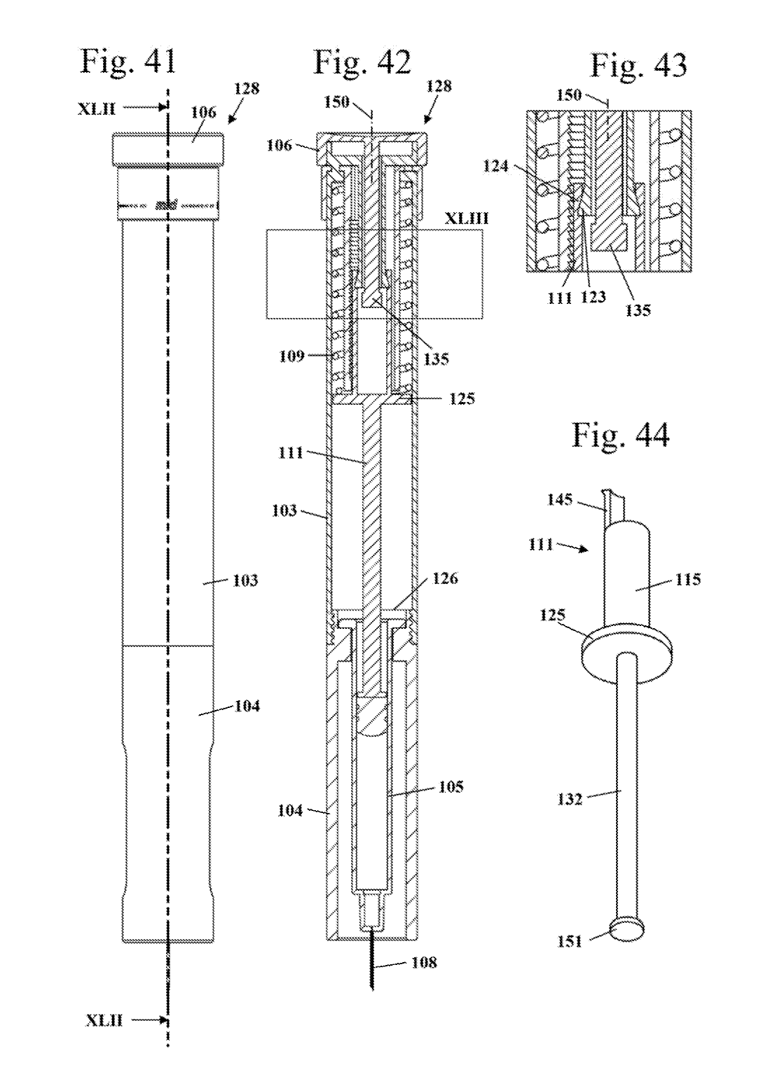

In order for an injection to be released, the operating button 106 is displaced in the proximal direction 131. The proximal terminal position of the operating element 106 is shown in FIGS. 41 to 43. In this position of the operating element 106, the injection device 101 is in a starting position 128 at which the injection commences. As is shown in FIGS. 42 and 43, the support portion 135 by virtue of the proximal movement of the operating element 106 has been moved in the proximal direction out of the region of the locking arms 122. The locking arms 122 on account thereof can pivot in a radially inward manner and thus be moved out of the locking depression 124. A ramp which by virtue of the force exerted by the injection spring 9 exerts a force component in a radially inward manner on the locking protrusions 123 is provided on the locking depression 124. The locking protrusions 123 are accordingly chamfered. The injection spring 109 pushes the metering piston 111 in the proximal direction. On account thereof, the locking depression 124 is moved in the proximal direction and deflects the locking protrusions 123 in a radially inward manner. On account thereof, the injection spring 109 can push the metering piston 111 in the proximal direction. The latching installation 142 herein is active in the set rotary position 152, 153, 154. In the case of the injection device 101, the desired injection rate is to be set prior to the beginning of the injection at the setting ring 117. After the injection rate has been set, the operating element 106 is pushed in the proximal direction 131, the injection on account thereof being automatically released.

FIGS. 44 to 49 show the metering piston 111 in detail. The piston rod 132 at the proximal end thereof supports a bearing disk 151 for bearing on the plug 110 of the container 105. As is shown in FIG. 47, the latching web 143 runs perpendicularly to the longitudinal central axis 50, thus at an angle δ in relation to the longitudinal central axis 150, the angle δ being 90°. The configuration of the locking depression 124 is also shown in FIG. 49. The locking depression 124 has an external wall which is inclined in relation to the longitudinal central axis 150 and which pushes the locking arms 122 (FIG. 43) in a radially inward manner. An encircling locking depression 124 is provided in the embodiment. However, a plurality of locking depressions 124 that are configured in a mutually separate manner can also be advantageous.

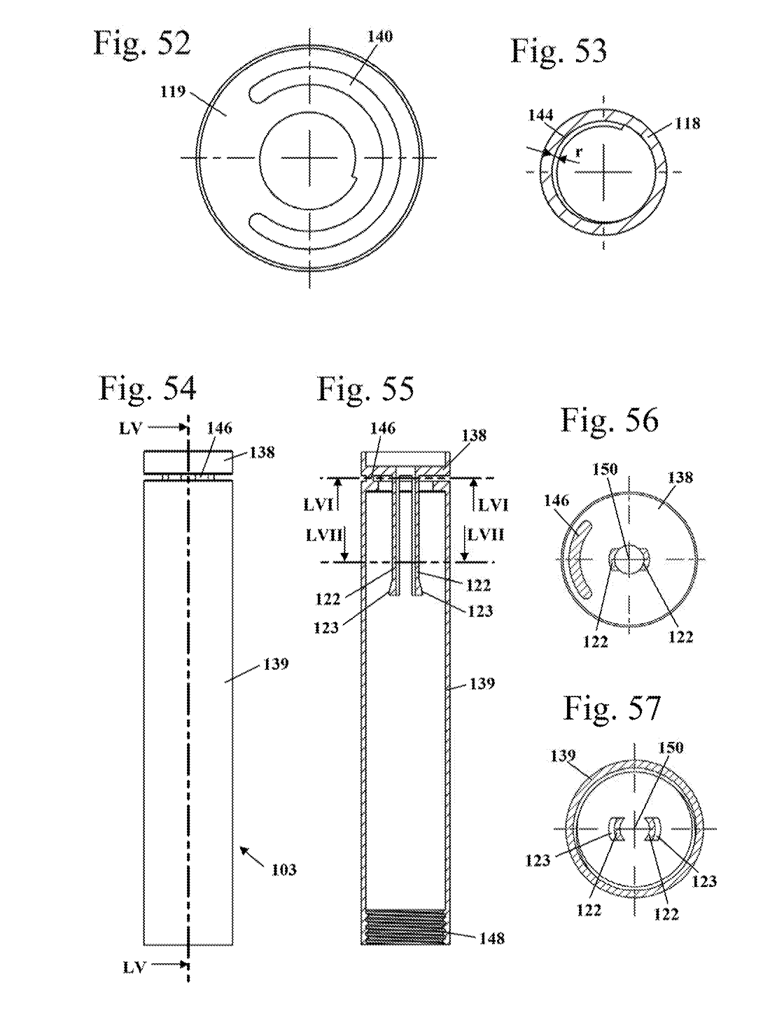

The setting part 116 is shown in FIGS. 50 to 53. The setting part 116 includes the sleeve portion 118 and the setting ring 117 which at the proximal end of the setting part 116 is connected to the sleeve portion 118 by way of a wall portion 119. The wall portion 119 has an opening 140, the upper housing part 103 protruding through the latter. As is also shown in FIG. 51, the latching depressions 144 are aligned so as to be perpendicular to the longitudinal central axis 150 (FIG. 50). FIGS. 52 and 53 also show the dissimilar depths of the latching depressions 144. The depth r of the latching depressions 144 decreases continuously in the circumferential direction. A stepless setting of the injection rate can be achieved in a simple manner on account thereof.

As is shown in FIGS. 54 and 55, the upper housing part 103 has a main body 139 to which a bearing portion 138 is fixed by way of a web 146. The bearing portion 138 is disposed on the distal side of the upper housing part 103 and serves for fixing the operating element 106. The bearing portion 138 and the main body 139 are advantageously configured so as to be mutually separate, and in the assembly of the setting part 116 are interconnected by way of the web 146. As is shown in FIGS. 55 to 57, the two locking arms 122 having the locking protrusions 123 are configured so as to be mutually mirror-symmetrical and are disposed in the interior of the main body 139. The web 146 extends across only approximately a quarter of the circumference. As is shown in FIG. 52, the opening 140 extends about the longitudinal central axis 150 across approximately three quarters of a complete circle. On account thereof, the setting part 116 can be rotated in relation to the upper housing part 103 by approximately 180° about the longitudinal axis 150.

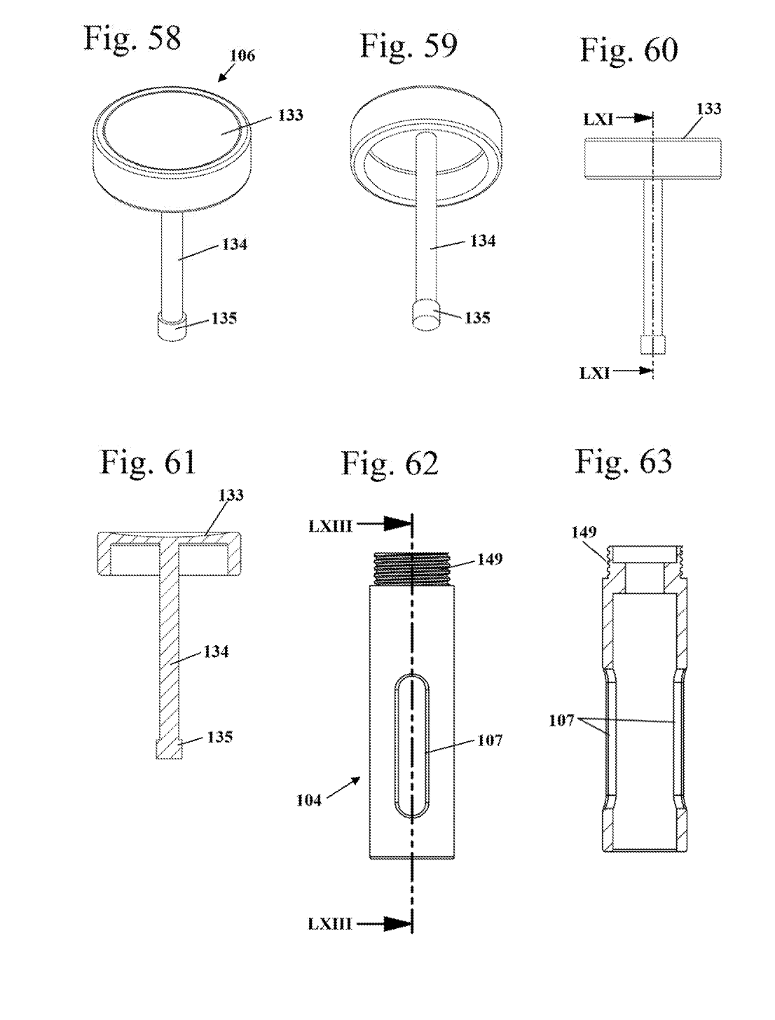

As is shown in FIGS. 58 to 61, the operating element 106 has a distal operating portion 133 at which the operator can push the operating element 106 in the proximal direction. The operating portion 133 is connected to the support portion 135 by way of a bar portion 134 which protrudes in the proximal direction. The bar portion 134 and the support portion 135 are configured as a cylinder, wherein the bar portion 134 has an external diameter that is smaller than that of the support portion 135. On account thereof, the locking arms 122 can deflect in a radially inward manner when the locking protrusions 123 are located so as to be adjacent to the bar portion 134.

FIGS. 62 and 63 show the lower housing part 104 having the viewing windows 107. The lower housing part 104 on the distal side thereof has an external thread 149 which interacts with an internal thread 148 (FIG. 56) of the upper housing part 103 and conjointly with the latter forms a threaded connection 147 (FIG. 30) by way of which the two housing parts 103, 104 are fixedly interconnected.

It is understood that the foregoing description is that of the preferred embodiments of the invention and that various changes and modifications may be made thereto without departing from the spirit and scope of the invention as defined in the appended claims.

What is claimed is:

1. An injection device for automatically squeezing out a dosage of injection liquid from a container thereof, the injection device comprising:
said container configured to hold the injection liquid;
a housing defining a longitudinal central axis;
a first component;
a second component;
wherein said first component and said second component move relative to one another when the injection liquid is squeezed out;
an injection spring configured to store energy and to, when the injection liquid is squeezed out from said container, at least partially release said stored energy to cause the dosage of the injection liquid to be squeezed out from said container;
a setting device to set an injection rate at which the injection liquid is squeezed out from said container;
said setting device being configured to influence the energy required for moving said second component in relation to said first component;
said first component and said second component being moveable relative to one another in the direction of said longitudinal central axis when the injection liquid is squeezed out; and,
said setting device being configured, when setting the injection rate, to act on a rotary position of said first component in relation to said second component.

2. The injection device of claim 1, wherein said setting device enables a stepless setting of said injection rate.

3. The injection device of claim 1 further comprising:
a setting spring;
said setting device including an operating element; and,
said setting spring being configured to pretension said operating element in a direction counter to an increase in said injection rate.

4. The injection device of claim 1, wherein:
said setting device has a latching installation configured to act between said first component and said second component; and,
said latching installation in a first relative rotary position of said first component and of said second component has a higher force for overcoming latching than in a second relative rotary position of said first component and of said second component.

5. The injection device of claim 4, wherein:
said latching installation includes at least one latching element which interacts with at least one counter latching element;
said at least one latching element and said at least one counter latching element conjointly define a latching depth with which said at least one latching element and said at least one counter latching element overlap; and,
said latching depth is greater in said first relative rotary position than in said second relative rotary position.

6. The injection device of claim 5, wherein said latching element is configured as a latching web which protrudes into a latching depression that forms said counter latching element.

7. The injection device of claim 1, wherein said setting device includes a threaded connection that is formed between said second component and said housing; and, for setting said injection rate, said second component is configured to be moved by an operator in relation to said housing in the direction of said longitudinal central axis and by virtue of said threaded connection is rotated in relation to said housing.

8. The injection device of claim 7 further comprising:
a latching element running helically about said longitudinal central axis;
said threaded connection defining a first pitch angle; and,
said latching element defining a second pitch angle corresponding to said first pitch angle of said threaded connection.

9. The injection device of claim 1 further comprising:
a setting ring configured to be rotated about said longitudinal central axis for setting said injection rate;
the injection device defining a proximal direction;
said second component being connected to said setting ring which, for setting said injection rate, is to be rotated by an operator about said longitudinal central axis; and,
an operating element configured to be displaced in said proximal direction for releasing an injection.

10. The injection device of claim 1, wherein said first component is, when the injection liquid is squeezed out, guided so as to be movable in the direction of said longitudinal central axis and so as to be rotationally fixed in said housing.

11. The injection device of claim 1, wherein:
said second component is a setting part of the injection device; and,
said first component is a metering piston which is displaced in the direction of said longitudinal central axis when the injection liquid is squeezed out.

12. The injection device of claim 5, wherein said setting device includes a threaded connection that is formed between said second component and said housing; and, for setting said injection rate, said second component is configured to be moved by an operator in relation to said housing in the direction of said longitudinal central axis and by virtue of said threaded connection is rotated in relation to said housing.

13. The injection device of claim 12, wherein:
said latching element runs helically about said longitudinal central axis;
said threaded connection defines a first pitch angle; and,
said latching element defines a second pitch angle corresponding to said first pitch angle of said threaded connection.
